(19) United States
(12) Patent Application Publication
(10) Pub. No.: US 2007/0151479 A1
(43) Pub. Date: Jul. 5, 2007

(54) Acidic monoazo dyestuffs

(76) Inventor: Ludwig Hasemann, Mullheim-Niederweiler (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 10/585,231

(22) PCT Filed: Dec. 23, 2004
(86) PCT No.: PCT/IB04/04292

§ 371 Date: Jun. 30, 2006

(30) Foreign Application Priority Data
Jan. 5, 2004 (EP) 0400057.2

(51) Int. Cl. C09D 11/00; C07D 251/44

(52) U.S. Cl. 106/31.48; 534/622

(57) ABSTRACT

Disclosed are novel dyestuff of the formula (I) 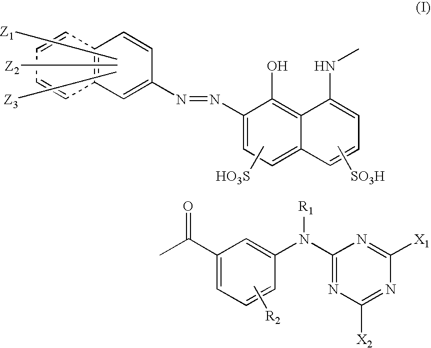 wherein all substituents are defined in the claims. These dyestuffs are useful for printing or dyeing substrates, especially textile fibre materials, paper and papery substrates and plastic films and plastic transparencies.

The invention relates to novel compounds, the use of such compounds and/or mixtures thereof as dyestuffs for printing recording materials, especially paper or papery substrates, textile fibre materials, plastic films and plastic transparencies by the inkjet printing process and also to the recording materials printed thereby.

Inkjet printing processes are becoming more and more important for industrial applications.

Inkjet printing processes are known. In what follows, the principle of inkjet printing will only be discussed very briefly. Details of this technology are described for example in the Ink-Jet-Printing section of R. W. Kenyon in “Chemistry and Technology of Printing and Imaging Systems”, Peter Gregory (editor), Blackie Academic & Professional, Chapmann & Hall 1996, pages 113-138, and references cited therein.

Together with more recently developed ink-jet ink receiving layers comprising micro-porous or porous surfaces the need for oxidation resistant dyestuff has become more important. The problem solved by the dyestuffs of the present invention is to provide dyestuffs resistant to oxidation and especially to increase resistance to ozone. This problem has been solved by providing dyestuffs according to the invention.

This invention provides dyestuff of formula (I) 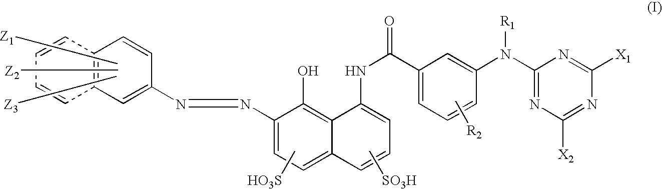
wherein
R₁ is H; C₁₋₄alkyl; substituted C₁₋₄alkyl; phenyl or substituted phenyl,
R₂ is H; C₁₋₄alkyl; substituted C₁₋₄alkyl; C₁₋₄alkoxy; —COOH; —COOCH₃; —CF₃; —SO₃H, —CN or SO₂NHR₆,
   where R₆ is H, C₁₋₄ Alkyl, phenyl or substituted phenyl and
X₁ is NR₃R₄; SR₅; OH;
X₂ is NR₃R₄; SR₅; OH;
   wherein
R₃ is H, C₁₋₄alkyl; substituted C₁₋₄alkyl; substituted phenyl, naphthyl or substituted naphthyl
R₄ is H; C₁₋₄alkyl; substituted C₁₋₄alkyl; substituted phenyl, naphthyl or substituted naphthyl
or R₃ and R₄ form a 5- or 6-membered ring containing one or two hetero atoms, in addition to N, O or S, which heterocyclic ring is unsubstituted or substituted by one or two C₁₋₄alkyl groups
R₅ is C₁₋₄alkyl; substituted C₁₋₄alkyl; phenyl or substituted phenyl and X₁ has not the meaning of X₂ unless X₁ or X₂ signifies SR₅ or OH; and
Z₁ is H; C₁₋₄alkyl; substituted C₁₋₄alkyl; C₁₋₄alkoxy; —OH; —COOH; —COOCH₃; —CF₃; —SO₃H ; amino; alkylamino, —CN or SO₂NHR′₆, where R′₆ is H, C₁₋₄ alkyl, phenyl or substituted phenyl
Z₂ is H; C₁₋₄alkyl; substituted C₁₋₄alkyl; C₁₋₄alkoxy; OH; COOH; —SO₃H
Z₃ is H, C₁₋₄alkyl; substituted C₁₋₄alkyl; C₁₋₄alkoxy; OH; COOH; —SO₃H as free acid or in salt form, as well as mixtures thereof.

Preferred compounds of formula (I) are characterized in that
R₁ is H; C₁₋₄alkyl; substituted C₁₋₄alkyl,
R₂ is H; C₁₋₄alkyl; substituted C₁₋₄alkyl; C₁₋₄alkoxy; —COOH or —SO₃H and
X₁ is NR₃R₄; SR₅; OH;
X₂ is NR₃R₄; SR₅; OH;
   wherein
R₃ is H, C₁₋₄alkyl; substituted C₁₋₄alkyl; substituted phenyl, naphthyl or substituted naphthyl
R₄ is H; C₁₋₄alkyl; substituted C₁₋₄alkyl; substituted phenyl, naphthyl or substituted naphthyl or
R₃ and R₄ form a 5- or 6-membered ring containing one or two hetero atoms, in addition to N, O or S, which heterocyclic ring is unsubstituted or substituted by one or two C₁₋₄alkyl groups
R₅ is C₁₋₄alkyl; substituted C₁₋₄alkyl; phenyl or substituted phenyl and X₁ has not the meaning of X₂ unless X₁ or X₂ signifies SR₅ or OH; and
Z₁ is H; C₁₋₄alkyl; substituted C₁₋₄alkyl; C₁₋₄alkoxy; —OH; —COOH; —COOCH₃; —CF₃; —SO₃H; amino; alkylamino, —CN or SO₂NHR′₆, where R′₆ is H, C₁₋₄ alkyl, phenyl or substituted phenyl
Z₂ is H; C₁₋₄alkyl; substituted C₁₋₄alkyl; C₁₋₄alkoxy; OH; COOH; —SO₃H
Z₃ is H, C₁₋₄alkyl; substituted C₁₋₄alkyl; C₁₋₄alkoxy; OH; COOH; —SO₃H as free acid or in salt form, as well as mixtures thereof

Preferred compounds according to formula (I) have the formula (Ia) 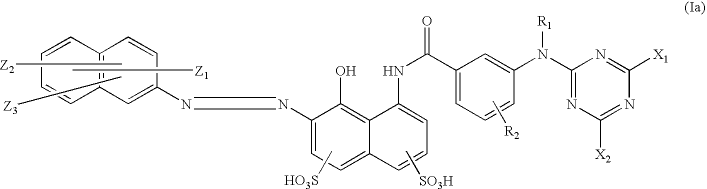
wherein
R₁ is H; C₁₋₄alkyl; substituted C₁₋₄alkyl,
R₂ is H; C₁₋₄alkyl; substituted C₁₋₄alkyl; C₁₋₄alkoxy; —COOH or —SO₃H and
X₁ is NR₃R₄; SR₅; OH;
X₂ is NR₃R₄; SR₅; OH;
   wherein
R₃ is H, C₁₋₄alkyl; substituted C₁₋₄alkyl; substituted phenyl, naphthyl or substituted naphthyl
R₄ is H; C₁₋₄alkyl; substituted C₁₋₄alkyl; substituted phenyl, naphthyl or substituted naphthyl
R₅ is C₁₋₄alkyl; substituted C₁₋₄alkyl; phenyl or substituted phenyl
   wherein
R₃ is H, C₁₋₄alkyl; substituted C₁₋₄alkyl; substituted phenyl, naphthyl or substituted naphthyl
R₄ is H; C₁₋₄alkyl; substituted C₁₋₄alkyl; substituted phenyl, naphthyl or substituted naphthyl or
R₃ and R₄ form a 5- or 6-membered ring containing one or two hetero atoms, in addition to N, O or S, which heterocyclic ring is unsubstituted or substituted by one or two C₁₋₄alkyl groups
R₅ is C₁₋₄alkyl; substituted C₁₋₄alkyl; phenyl or substituted phenyl and X₁ has not the meaning of X₂ unless X₁ or X₂ signifies SR₅ or OH; and
Z₁ is H; C₁₋₄alkyl; substituted C₁₋₄alkyl; C₁₋₄alkoxy; —OH; —COOH; —COOCH₃; —CF₃; —SO₃H; amino; alkylamino, —CN or SO₂NHR′₆, where R′₆ is H, C₁₋₄ alkyl, phenyl or substituted phenyl
Z₂ is H; C₁₋₄alkyl; substituted C₁₋₄alkyl; C₁₋₄alkoxy; OH; COOH; —SO₃H
Z₃ is H, C₁₋₄alkyl; substituted C₁₋₄alkyl; C₁₋₄alkoxy; OH; COOH; —SO₃H as free acid or in salt form, as well as mixtures thereof

Further more preferred compounds according to formula (I) have the formula (Ib) 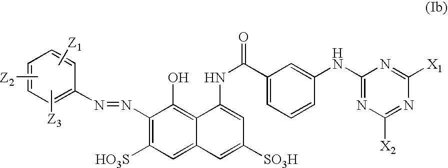
wherein
R₁ is H; C₁₋₄alkyl; substituted C₁₋₄alkyl,
R₂ is H; C₁₋₄alkyl; substituted C₁₋₄alkyl; C₁₋₄alkoxy; —COOH or —SO₃H and
X₁ is NR₃R₄; SR₅; OH;
X₂ is NR₃R₄; SR₅; OH;
   wherein
R₃ is H, C₁₋₄alkyl; substituted C₁₋₄alkyl; substituted phenyl, naphthyl or substituted naphthyl
R₄ is H; C₁₋₄alkyl; substituted C₁₋₄alkyl; substituted phenyl, naphthyl or substituted naphthyl or
R₃ and R₄ form a 5- or 6-membered ring containing one or two hetero atoms, in addition to N, O or S, which heterocyclic ring is unsubstituted or substituted by one or two C₁₋₄alkyl groups
R₅ is C₁₋₄alkyl; substituted C₁₋₄alkyl; phenyl or substituted phenyl and X₁ has not the meaning of X₂ unless X₁ or X₂ signifies SR₅ or OH; and
Z₁ is H; C₁₋₄alkyl; substituted C₁₋₄alkyl; C₁₋₄alkoxy; —OH; —COOH; —COOCH₃; —CF₃; —SO₃H ; amino; alkylamino, —CN or SO₂NHR′₆, where R′₆ is H, C₁₋₄ alkyl, phenyl or substituted phenyl
Z₂ is H; C₁₋₄alkyl; substituted C₁₋₄alkyl; C₁₋₄alkoxy; OH; COOH; —SO₃H
Z₃ is H, C₁₋₄alkyl; substituted C₁₋₄alkyl; C₁₋₄alkoxy; OH; COOH; —SO₃H as free acid or in salt form, as well as mixtures thereof.

The preferred compounds of the formula (I), (Ia) or (Ib) don't have fiber reactive groups or groups which are reactive to the substrate to which they are applied.

The preferred compounds of the formula (I), (Ia) or (Ib) wherein the substituents R₃ and R₁₋₄ form together a 5- or 6-membered ring containing one hetero atoms, in addition to N, which heterocyclic ring is unsubstituted or substituted by one or two C₁₋₄alkyl groups. The additional heteroatom is by preference a O or a N atom, more preferred a O atom. By preference, this ring is 6-membered ring. By preference the additional heteroatom is a O atom. In the more preferred compounds wherein the substituents R₃ and R₄ form together a 5- or 6-membered ring —NR₃R₄ is a morpholino group.

In more preferred compounds of the formula (I), (Ia) or (Ib)
R₃ is H, C₁₋₄alkyl; substituted C₁₋₄alkyl; substituted phenyl, naphthyl or substituted naphthyl
R₄ is H; C₁₋₄alkyl; substituted C₁₋₄alkyl; substituted phenyl, naphthyl or substituted naphthyl.

Substituted phenyl or naphtyl means phenyl groups or naphtyl groups substituted by —SO₃H, —COOH, —OH, alkyl or alkoxy. These alkyl or alkoxy groups are by preference C₁₋₄alkyl; C₁₋₄alkoxy which may be further substituted by —SO₃H, —COOH, —OH. Preferred alkylgroups are methyl or ethyl. Preferred alkoxygroups are methoxy or ethoxy.

Substituted alkyl means alkyl groups which are further substituted by groups selected from —OH, —COOH, —NH₂, —NHalkyl, —N(alkyl)₂, —SO₃H, —O-alkyl. Alkyl groups may also be branched.

The deprotonable groups as for example the —COOH, or —SO₃H groups may also be in the salt forms e.g. —COOM, or —SO₃M. it is also possible that only a part of the protons of those deprotonable groups are neutralized. Furthermore several different cations may be present, thus the dyestuffs are in a mixed salt form.

Suitable cations M are alkali metal, alkaline earth metal, ammonium, alkanolammonium or alkylammonium cations. Examples of corresponding cations are the sodium, lithium or ammonium cations or mono-, di- or triethanolammonium cations.

Examples of such cations are alkali metal cations, e.g. lithium, sodium, potassium, and ammonium cations or substituted ammonium cations, e.g. mono-, di-, tri- and tetra-methylammonium, tri-ethylammonium, and mono-, di-, and tri-ethanolammonium. The preferred cations are the alkali metal cations and the ammonium cation, with the sodium cation being the most preferred.

A further embodiment of the present invention is the preparation of compounds of formula (I) characterized in that in a first step a compound of formula (II) 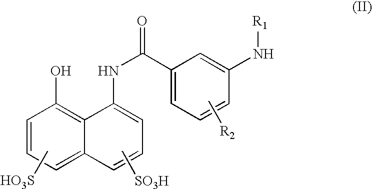
wherein all substituents have the meanings as defined above are reacted with a compound of formula (III)

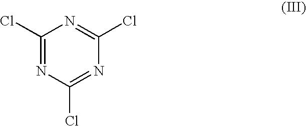

The reaction leads to compounds according to formula (IV)

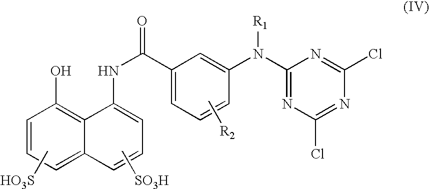

In a second step the product of formula (IV) is reacted with one part of a compound of formula HX₁ wherein X₁ has the formula as described above which leads to compound of formula (V)

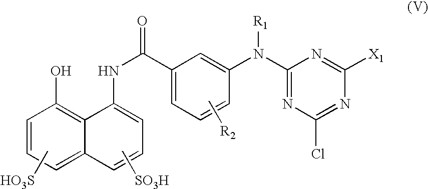

In a third step compound of formula (V) are condensated with a compound of formula HX₂ wherein X₂ has the formula as described above leading to compound of formula (VI) 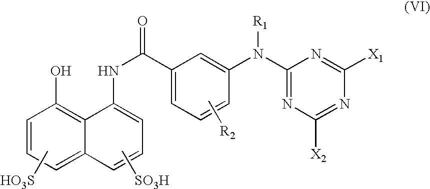
wherein substituents R₁ and R₂ have the same meanings as defined above.

In the final step a compound of formula (VI) is coupled with the diazoniumsalt of a compound of formula (VII) 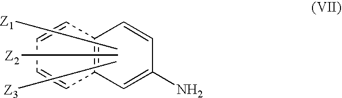
leading to the dyestuff of formula (I) 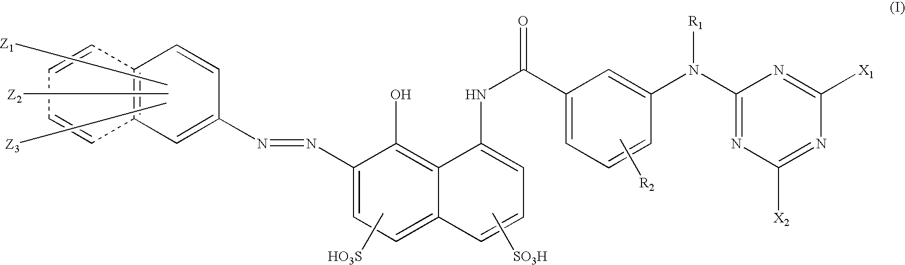
wherein all substituents have the same meanings as defined above.

The diazoniumsalt of the compound of formula (VII) means either a compound of formula (VIIa) or a compound of formula (VIIb)

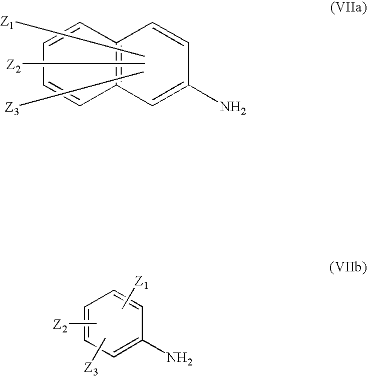

In the procedure of producing the compounds the starting compound of formula (II) can to be synthesized by known processes, e.g. condensation of 3-nitro-benzoylchloride with H-Acid and subsequent reduction.

In the first step of the abovementioned reaction the temperature is in the range of 0° C. to 40° C., preferably 0° C. to 25° C. and the pH is in the range of 2 to 6, preferably 3 to 5. In the second step of the abovementioned reaction the temperature is in the range of 20-70° C., preferably 30 to 60° C. and the pH is in the range of 3 to 9, preferably 5 to 8. In the third step of the abovementioned reaction the temperature is in the range of 40-95° C., preferably 40 to 85° C. and the pH is in the range of 5 to 10, preferably 6 to 9.

Any alkyl or alkylene group starting from C₃-alkyl or C₃-alkylene may be linear or branched. Any alkoxy group starting from C₃-alkoxy may be linear or branched.

Useful cations for salt formation include in particular alkali metal, alkaline earth metal as well ammonium cations. Examples of such cations are alkali metal cations, for example potassium, lithium or sodium ions and ammonium cations, e.g. mono-, di-, tri- or tetra-methyl ammonium cations or mono-, di-, tri- or tetra-ethyl ammonium cations or mono-, di- or tri-ethanol ammonium cations. The cations may be the same or different, i.e. the compounds may be in mixed salt-form.

The dyestuffs according to the invention may be used alone or for shading other dyestuffs or dyestuff mixtures. The compounds (I) according to the invention may be mixed with other colorants to match exactly the desired hue, thus the colorants according to the invention. In principle any other colorant which is compatible with the compounds according to the invention may be used for this purpose.

On the other hand the compounds according to the invention having the formula (I) may itself be used as shading component for shading other colorants.

The mixtures according to the invention itself may be used as a shading component and blended with other compatibles dyestuff mixtures to achieve the desired shade.

Instead of blending the dyestuff to the mixtures it is also possible to prepared inks as described below and mixed these inks of different shades to achieve the desired colour.

The dyestuff can also formulated into liquid compositions. Stable liquid compositions may be achieved by desalting and concentrating a dyestuff solution by passing them through separation membranes such as cellulose acetate membrane or polybenzimidazolone membrane (PBIL).

Alternatively stable solution may be produced by mixing with amines, especially with mono-, di- or tri-alkanol amines or polyethoxylated amines. Those polyethoxylated amines are normally produced by reacting ethylene oxide or propylene oxide or mixtures thereof with mono-, di- or tri-alkanol amines or with alkanol derivatives of polyamines.

The dyestuff according to the invention may also used alone for printing. Thus in certain cases no shading dyestuffs are needed, for example if the shade is already correct. The amount of shading colorants are present in this mixtures is dependent on the shade that should be achieved. For example the shaded mixtures of dyes may comprise 50-99 wt-% of at least one compound according to formula (I) and 1-50 wt-% of at least one compound described as shading component as described and listed below, but not a compound according to formula (I).

By preference the shading component is present in an amount of 0.001 to 5% by weight, preferably 0.01 to 1% by weight, based on the total weight of the dry dye mixture.

For example the shading colorant may be selected (among others) from the group consisting of C.I. Direct Red 1, 11, 37, 62, 75, 81, 87, 89, 95, 227; C.I. Acid Red 115, 131, 144, 152, 186, 245, C.I. Pigment Red 122, 176, 184, 185 and 269.

In preferred mixtures, compounds according to formula (I) are mixed with at least one compound selected from C.I. (Colour Index) Acid Red 50, C.I. Acid Red 51, C.I. Acid Red 52, C.I. Acid Red 87, C.I. Acid Red 91, C.I. Acid Red 92, C.I. Acid Red 93, C.I. Acid Red 94, C.I. Acid Red 95, C.I. Acid Red 98 and C.I. Acid Red 289.

In further preferred mixtures, compounds according to formula (I) are mixed with at least one compound selected from C.I. Acid Red 1, C.I. Acid Red 33, C.I. Acid Red 35, C.I. Acid Red 40, C.I. Acid Red 76, C.I. Acid Red 106, C.I. Acid Red 138, C.I. Acid Red 155, C.I. Acid Red 160, C.I. Acid Red 172, C.I. Acid Red 249, C.I. Acid Red 264 and C.I. Acid Red 265.

In further preferred mixtures, compounds according to formula (I) are mixed with at least one compound selected from C.I. Acid Red 15, C.I. Acid Red 19, C.I. Acid Red 29, C.I. Acid Red 60, C.I. Acid Red 68, C.I. Acid Red 154 and C.I. Acid Red 176.

In further preferred mixtures, compounds according to formula (I) are mixed with at least one compound selected from C.I. Acid Red 30, C.I. Acid Red 34, C.I. Acid Red 37, C.I. Acid Red 42, C.I. Acid Red 54, C.I. Acid Red 57, C.I. Acid Red 231, C.I. Acid Red 266, C.I. Acid Red 301 and C.I. Acid Red 337

Especially preferred mixture comprises a compound according to formula (I) and C.I. Acid Red 52 and/or C.I. Acid Red 289 which have the following formulae

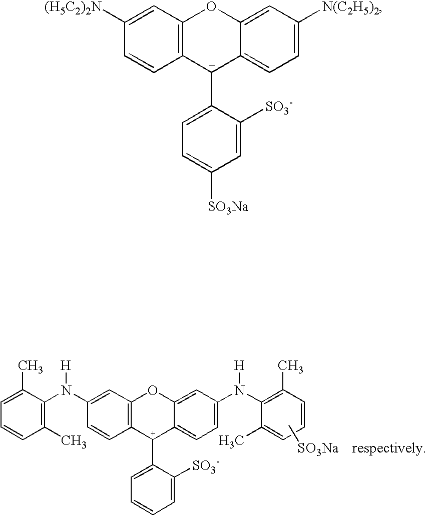

It is possible to use the dyestuff (I) without any further dyestuff in the composition Ink jet printing compositions according the present invention.

A further embodiment of the present invention relates to a composition for printing recording materials, preferably paper and papery substrates, textile fibre materials, plastic films and plastic transparencies by the inkjet printing process, comprising
1) the dye of the formula (I) as defined above and
2) water or a medium including a mixture of water and an organic solvent, an anhydrous organic solvent or a solid having a low melting point,
   The inkjet printing composition may optionally comprise further additives.

A further embodiment of the present invention relates to a inkjet printing composition for printing recording materials, preferably paper and papery substrates, textile fibre materials, plastic films and plastic transparencies, comprising
1) a mixture of dyestuffs as defined above and
2) water or a medium including a mixture of water and an organic solvent, an anhydrous organic solvent or a solid having a low melting point,
   The inkjet printing composition may optionally comprise further additives.

A further embodiment of the invention relates to the use of the above mentioned compositions for the ink-jet printing process.

By additionally disposing at least one nozzle with yellow, magenta or cyan ink side by side it is possible to obtain colour reproductions in high quality. This process is known as polychromatic printing or, when three colour components are used, as trichromatic printing.

The dyestuffs and the composition of the invention can be used with all known and suitable inkjet printers for printing paper or papery substrates, textile fibre materials, plastic films and plastic transparencies. This applies not only to the use in monochromatic printing but also to polychromatic printing, especially trichromatic printing.

The composition of the ink for the inkjet printing process has to possess a suitable conductivity, sterility in storage, viscosity and surface tension to meet the specific requirements of inkjet ink. In addition, the prints on the recording materials have to have good properties and fastness.

Useful recording materials, as mentioned above, are preferably paper and papery substrates, textile fibre materials, plastic films and plastic transparencies. But glass and metal may be used as well.

Useful papers or papery substrates include all known such materials. Preference is given to papers or papery substrates coated on at least one side with a material which is particularly receptive to ink compositions. Such papers or papery materials are described inter alia in DE 3018342, DE 4446551, EP 164196 and EP 875393.

Useful textile fibre materials are in particular hydroxyl-containing fibre materials. Preference is given to cellulosic fibre materials, which consist of or comprise cellulose. Examples are natural fibre materials such as cotton, linen or hemp and regenerated fibre materials such as, for example, viscose and also lyocell.

Useful plastic films or plastic transparencies include all known such materials. Preference is given to plastic films or plastic transparencies coated on at least one side with a material which is particularly receptive to the ink compositions. Such plastic films or plastic transparencies are described inter alia in EP 755332, U.S. Pat. No. 4,935,307, U.S. Pat. No. 4,956,230, U.S. Pat. No. 5,134,198 and U.S. Pat. No. 5,219,928.

Further possible and useful ink jet recording elements which receive the dyestuffs or the ink compositions respectively comprise a support having thereon an image-receiving layer of micro-porous polymeric particles or porous polymeric particles or micro-porous inorganic particles or porous inorganic particles. The particles may be held together by a (polymeric) binder.

The dyes of the formula (I) used in the inks should preferably be low in salt, i.e. have a total salt content of less than 0.5% by weight, based on the weight of the dyes. Dyes having higher salt contents (owing to their preparation and/or the subsequent addition of extenders) may be desalted, for example by means of membrane separation processes, such as ultrafiltration, reverse osmosis or dialysis.

The inks preferably include a total amount of dyes which is in the range from 0.5 to 35% by weight, preferably in the range from 1 to 35% by weight, more preferably in the range from 2 to 30% by weight, most preferably in the range from 2.5 to 20% by weight, based on the total weight of the ink.

The inks include 99.5-65% by weight, preferably 99-65% by weight, more preferably 98-70% by weight, most preferably 97.5-80% by weight, of an abovementioned medium 2), which includes a mixture of water and an organic solvent, an anhydrous organic solvent or a solid having a low melting point.

When said medium 2) is a mixture including water and an organic solvent or an anhydrous organic solvent, the dye mixtures comprising at least one compounds of formula (I) are preferably completely dissolved in this medium.

Preferably the dye mixtures comprising at least one compounds of formula (I) have a solubility of not less than 2.5% by weight in this medium 2) at 20° C.

When the ink composition of the invention is used for printing paper or papery substrates, the inks are preferably used together with the following compositions.

When the medium is a mixture of water and an organic solvent, the weight ratio of water to organic solvent is preferably in the range from 99:1 to 1:99, more preferably in the range from 99:1 to 50:50, particularly preferably in the range from 95:5 to 80:20.

It is preferable for the organic solvent, which is included in the mixture with water to be a water-soluble solvent or a mixture of various water-soluble solvents. Preferred water-soluble organic solvents are C₁₋₆-alcohols, preferably methanol, ethanol, n-propanol, isopropanol, n-butanol, sec-butanol, tert-butanol, n-pentanol, cyclopentanol and cyclohexanol; linear amides, preferably dimethylformamide, or dimethylacetamide; ketones and keto alcohols, preferably acetone, methyl ethyl ketone, cyclohexanone and diacetone alcohol; water-miscible ethers, preferably tetrahydrofuran and dioxane; diols, preferably diols possessing 2 to 12 carbon atoms, e.g. 1,5-pentanediol, ethylene glycol, propylene glycol, butylene glycol, pentylene glycol, hexylene glycol and thiodiglycol and oligo- and poly-alkylene glycols, preferably diethylene glycol, triethylene glycol, 1,2-propylenglycol, polyethylene glycol and polypropylene glycol; triols, preferably glycerol and 1,2,6-hexanetriol; mono-C₁₋₄-alkyl ethers of diols, preferably mono-C₁₋₄-alkyl ethers of diols possessing 2 to 12 carbon atoms, particularly preferably 2-methoxyethanol, 2-(2-methoxyethoxy)ethanol, 2-(2-ethoxyethoxy)ethanol, 2-[2-(2-methoxyethoxy)ethoxy]-ethanol, 2-[2-(2-ethoxyethoxy)ethoxy]ethanol, diethylenglycol-mono-n-butylether, ethylene glycol monoallyl ether and polyoxyethylenalkylether (for example Emulgen 66 of KAO Corp. (Emulgen is a trade mark of the Kao Corp.)); alkanolamines, preferably 2-diethylamine-1-ethanol, 3-dimethylamine-1-propanol, 3-diethylamine-1-propanol, 2-(2-aminoethoxy)ethanol, 2-(2-dimethylaminoethoxy)-ethanol, 2-(2-diethylaminoethoxy)ethanol, mono-, di-, triethanolamine, monoglycolamines and polyglycolamines, which may be obtained by reaction of ammonia, alkyl- or hydroxyalkylamines like methyl amine, ethyl amine, dimethyl amine, diethylamine, mono-, di- and triethanolamines with alkyleneoxides for example ethylenoxide, 1,2-propylenoxide, 1,2-butylenoxide or 2,3-butylenoxide in suitable ratios as described in DE2061760A, preferably diethylenglycolamine, triethylenglycolamin, Bis-diethylenglycolamin, polyoxyethylen-(6)-triethanolamine, polyoxyethylen-(9)-triethanolamine, o-(2-aminoethyl)-polyethylenglycol 750, o,o-Bis-(2-aminopropyl)-polyethylenglycol 500, 800, 1900, 2000, o,o′-Bis-(3-aminopropyl)-polyethylenglycol 1500, cyclic amides, preferably 2-pyrrolidone, N-methyl-2-pyrrolidone, N-ethyl-2-pyrrolidone, N-(2-hydroxy)ethyl-2-pyrrolidone, caprolactam and 1,3-dimethylimidazolidone; cyclic esters, preferably caprolactone; sulphoxides, preferably dimethyl sulphoxide and sulpholane.

In a preferred composition, the medium as per 2) includes water and at least 2 or more, more preferably 2 to 8, water-soluble organic solvents.

Particularly preferred water-soluble solvents are cyclic amides, particularly 2-pyrrolidone, N-methyl-2-pyrrolidone and N-ethyl-2-pyrrolidone; N-(2-hydroxy)ethyl-2-pyrrolidone, C₁₋₆-alcohols, preferably n-propanol, cyclohexanol, diols, preferably 1,5-pentanediol, ethylene glycol, thiodiglycol, diethylene glycol, triethylene glycol and 1,2-propyleneglycol, triols, preferably glycerol; and mono-C₁₋₄-alkyl and C₁₋₄-alkyl ethers of diols, more preferably mono-C₁₋₄-alkyl ethers of diols possessing 2 to 12 carbon atoms, particularly preferably 2-[2-(2-methoxyethoxy)-ethoxy]-ethanol, diethylenglycol-mono-n-butylether, (for example Emulgen 66 of KAO Corp. (Emulgen is a trade mark of the Kao Corp.)), 2-diethylamine-1-ethanol, 3-dimethylamine-1-propanol, 3-diethylamine- 1-propanol, 2-(2-diethylaminoethoxy)-ethanol, triethanolamine, diethylenglycolamin, polyglycolamines, preferably polyoyxethylen-(6) -triethanolamin, polyoxyethylen-(9)-triethanolamine, o-(2-aminoethyl)-polyethylen 750, o,o-bis-(2-aminopropyl)-poyethylen 500 and o,o-bis-(3-aminopropyl)-polyethylenglycol 1500.

A preferred medium as per 2) comprises
(a) 75 to 95 parts by weight of water and
(b) 25 to 5 parts of one or more of the watersoluble solvents. wherein the parts are by weight and all parts of (a) and (b) add up to 100.

Examples of further useful ink compositions including water and one or more organic solvents are found in the Patent Specifications U.S. Pat. No. 4,963,189, U.S. Pat. No. 4,703,113, U.S. Pat. No. 4,626,284 and EP 425150A.

When the medium as per 2) includes an anhydrous (i.e. less than 1% by weight of water) organic solvent, this solvent will have a boiling point of 30 to 200° C., more preferably of 40-150° C., particularly preferably of 50 - 125° C.

The organic solvent can be water-insoluble, water-soluble or mixtures of such solvents.

Preferred water-soluble organic solvents are all above-described water-soluble organic solvents and mixtures thereof.

Preferred water-insoluble solvents include inter alia aliphatic hydrocarbons; esters, preferably ethyl acetate; chlorinated hydrocarbons, preferably CH₂Cl₂; and ethers, preferably diethyl ether; and mixtures thereof.

When the liquid medium as per 2) includes a water-insoluble organic solvent, it is preferable to add a polar solvent to increase the solubility of the dye in the liquid medium.

Examples of such polar solvents are C₁₋₄-alcohols, preferably ethanol or propanol; ketones, preferably methyl ethyl ketone.

The anhydrous organic solvent can consist of a single solvent or a mixture of 2 or more different solvents.

When it is a mixture of different solvents, a mixture including 2 to 5 different anhydrous solvents is preferred. This makes it possible to provide a medium as per 2) which permits good control of the drying properties and of the stability of the ink composition in storage.

Ink compositions including an anhydrous organic solvent or mixtures thereof are of particular interest when rapid drying times are required and especially when they are used for prints on hydrophobic and non-absorbing substrates, such as plastic, metal and glass.

Preferred low-melting media have a melting point of 60 to 125° C. Useful low-melting solids include long-chain fatty acids or alcohols, preferably those having a C₁₈₋₂₄-carbon chain, and sulphonamides.

The ink composition of the invention may further include as auxiliaries additional components which are normally used in inkjet inks, for example viscosity improvers, surface tension improvers, biocides, corrosion inhibitors, levelling agents, drying agents, humefactants, ink penetration additives, light stabilizers, UV absorbers, optical brighteners, coagulation reducers, ionic or nonionic surfactants, conducting salts and pH buffers.

These auxiliaries are preferably added in an amount of 0-5% by weight.

To prevent precipitation in the ink compositions of the invention, the dyes used have to be purified clean. This can be done with commonly known purifying methods.

When printing textile fibre materials, useful additives, as well as the solvents, include water-soluble nonionic cellulose ethers or alginates.

The preferred material is paper. The paper may be plain or treated. Preference is given to ink compositions having a viscosity of 1 to 40 mpa's, especially 5 to 40 mPa-s, preferably 10 to 40 mPa's. Ink compositions having a viscosity of 10 to 35 mPa·s are particularly preferred.

Preference is given to ink compositions having a surface tension of 15-73 mN/m, especially 20-65 mN/m, particularly preferably 30-50 mN/m.

Preference is given to ink compositions having a conductivity of 0.1-100 mS/cm, especially 0.5-70 mS/cm, particularly preferably 1.0-60 mS/cm.

The inks may further include buffer substances, for example borax, borate or citrate. Examples are sodium borate, sodium tetraborate and sodium citrate.

They are used in particular in amounts of 0.1 to 3% by weight, preferably 0.1 to 1% by weight, based on the total weight of the ink, to set a pH of for example 5 to 9, especially 6 to 8. A citrate buffer is preferred in the case of alginatic inks.

The inks may further include customary additives, for example foam suppressants or especially fungal and/or bacterial growth inhibitors. These are customarily used in amounts of 0.01 to 1% by weight, based on the total weight of the ink. The printing inks and also the dye mixtures comprise at least the compound of formula (I).

The prints obtainable by the process of the invention have good general fastnesses, very good ozone-fastness , very good lightfastness and also sharp contours and a high colour strength. The inks provide prints of high optical density. Especially good brilliance together with a good ozone and light fastness is achieved with the dyes according to the invention.

The printing inks used are notable for good stability and good viscosity properties. The recording fluids of the invention have viscosity and surface tension values which are within the ranges suitable for the ink-jet processes. The viscosity remains virtually unchanged even in the event of high shearing forces occurring during printing. Recording fluids according to the invention in storage are not prone to the formation of precipitates that leads to fuzzy prints or nozzle cloggage.

A further aspect of the present invention is the use of the printing ink in trichromatic printing. Trichromatic printing is a very large application for all recording materials. This form of printing is normally carried out with a yellow, red and blue ink composition. Furthermore, the magenta dye mixtures of the invention may be used as an ink set in combination with black, yellow and/or cyan recording fluids.

This invention further provides recording materials, which have been printed with a composition according to the invention.

The compounds according to the invention may be used for dyeing cationic dyeable materials such as: homo- or mixed-polymers of acrylonitrile, acid modified polyester or polyamide; wool; leather including low affinity vegetable-tanned leather; cotton; bast fibers such as hemp, flax, sisal, jute, coir and straw; regenerated cellulose fibers, glass or glass products comprising glass fibers; and substrates comprising cellulose for example paper and cotton. They may also be used for printing fibers, filaments and textiles comprising any of the above mentioned materials in accordance with known methods. Printing may be effected by impregnation of the material to be printed with a suitable printing paste comprising one or more compounds of the present invention. The type of printing paste employed, may vary depending on the material to be printed. Choice of a suitable commercially available printing paste or production of a suitable paste, is routine for one skilled in the art. Alternatively, as already described, the compounds of the present invention may be used in the preparation of inks suitable for example for jet printing, in accordance with conventional methods as explained above.

Moreover, the dyestuffs are used for dyeing or printing of paper e.g., sized or unsized, wood-free or wood-containing paper or paper-based products such as cardboard. They may be used in continuous dyeing in the stock, dyeing in the size press, in a conventional dipping or surface coloring process. The dyeing and printing of paper is effected by known methods.

The dyeings and prints and particularly those obtained on paper, show good fastness properties.

The paper dyeings or printings made with the compounds according to the invention are clear and brilliant and have good light fastness. On exposure to light for a long time, the shade of the dyeing fades tone in tone. They show very good wet fastness properties; being fast to water, milk, fruit juice, sweetened mineral water, tonic water, soap and sodium chloride solution, urine etc. Furthermore, they have good alcohol fastness properties. The wet fastness properties are improved compared to known dyes showing otherwise similar properties. They do not exhibit a tendency towards two-sidedness. It is important to mention the extreme good fastness against ozon.

Paper dyed or printed with the compounds of the present invention can be bleached either oxidatively or reductively, a feature, which is important for the recycling of waste paper and old paper products.

The compounds of the present invention may also be used to dye paper containing wood-pulp where even dyeings, having good fastness properties are obtained. Furthermore, they may be used for the production of coated paper in accordance with known methods. Preferably when coating, a suitable filler, for example kaolin, is employed in order to give a one-side coated paper.

Moreover, the dye mixtures of the invention are useful as colorants in the electrophotographic toners and developers, for example one- and two-component powder toners, magnetic toners, liquid toners, polymerization toners and other specialty toners.

Typical toner binders are addition polymerization, polyaddition and polycondensation resins, such as styrene, styrene-acrylate, styrene-butadiene, acrylate, polyester, phenolic and epoxy resins, polysulfones, polyurethanes, individually or in combination, and also polyethylene and polypropylene, in or to which further ingredients, such as charge control agents, waxes or flow agents, may be present or added subsequently. Dye mixtures according to the invention are further useful as colorants in powders and powder coating materials, especially triboelectrically or electrostatically sprayed powder coating materials, which are used to coat the surfaces of articles made for example of metal, wood, plastic, glass, ceramic, concrete, textile material, paper or rubber. Powder coating resins employed are typically epoxy resins, carboxyl- and hydroxyl-containing polyester resins, polyurethane resins and acrylic resins together with customary curing agents. Combinations of resins are also used. For instance, epoxy resins are frequently used in combination with carboxyl- and hydroxyl-containing polyester resins.

The dye mixtures of the invention are also useful as colorants for color filters, for additive as well as subtractive color generation (P. Gregory “Topics in Applied Chemistry: High Technology Applications of Organic Colorants” Plenum Press, New York 1991, page 15-25), and also as colorants in electronic inks for electronic newspapers.

The examples hereinbelow illustrate the invention. Temperatures are in degrees Celsius; parts and percentages are by weight, unless otherwise stated.

### EXAMPLES OF INK COMPOSITIONS

A preferred ink composition according to the invention comprises
0.5-35 parts of a compound of formula (I),
65-99.5 parts of water or a medium including a mixture of water and an organic solvent, an anhydrous organic solvent or a solid having a low melting point and optionally
0-5 parts of one or more additives.

A more preferred ink composition according to the invention comprises
1-20 parts of a compound of formula (I),
80-99 parts of water or a medium including a mixture of water and an organic solvent, an anhydrous organic solvent or a solid having a low melting point and optionally
0-5 parts of one or more additives.

A particularly preferred ink composition according to the invention comprises
1-5 parts of a compound of formula (I),
95-99 parts of water or a medium including a mixture of water and an organic solvent, an anhydrous organic solvent or a solid having a low melting point and optionally
0-5 parts of one or more additives.

A more preferred ink composition according to the invention comprises
0.5-35 parts of a dye mixtures comprising at least one compound of formula (I) and at least C.I. Acid Red 52 or C.I. Acid Red 289, and
65-99.5 parts of water or a medium including a mixture of water and an organic solvent, an anhydrous organic solvent or a solid having a low melting point and optionally
0-5 parts of one or more additives.

A particularly preferred ink composition according to the invention comprises
1-20 parts of a dye mixtures comprising at least one compound of formula (I) and at least C.I. Acid Red 52 or C.I. Acid Red 289, and
80-99 parts of water or a medium including a mixture of water and an organic solvent, an anhydrous organic solvent or a solid having a low melting point and optionally
0-5 parts of one or more additives.

A further preferred ink composition according to the invention comprises
1-5 parts of a dye mixtures comprising at least one compound of formula (I) and at least C.I. Acid Red 52 or C.I. Acid Red 289, and
95-99 parts of water or a medium including a mixture of water and an organic solvent, an anhydrous organic solvent or a solid having a low melting point and optionally
0-5 parts of one or more additives.

The sum total of all the parts of the abovementioned compositions according to the invention is 100 parts.

The above mentioned composition is preferably prepared by heating the medium to 30 -40° C. and then adding a dye of a compound of formula (I) or a dye mixtures comprising at least one compound of formula (I) and at least C.I. Acid Red 52 or C.I. Acid Red 289. The composition is then cooled down to room temperature.

This ink composition is preferably used for printing papers or papery substrates.

The following Examples further serve to illustrate the invention, without restricting the scope of protection to these Examples. In the Examples all parts and all percentages are by weight, and the temperatures given are in degrees Celsius, unless indicated to the contrary.

### Example 1

a) Condensation of 1-(3′-Aminobenzoylamino)-8-Hydroxynaphthalin-3,6-Disulfonic Acid with Cyanurchloride

220 g of 1-(3′-aminobenzoylamino)-8-hydroxynaphthalin-3,6-disulfonic acid are solved with sodiumhydroxide in water. This solution were added to a suspension of 92.2 g of cyanurchloride in ice/water mixture, keeping the pH at 3 by adding sodiumhydroxide. After the termination of this reaction the compound of formula (VIII) 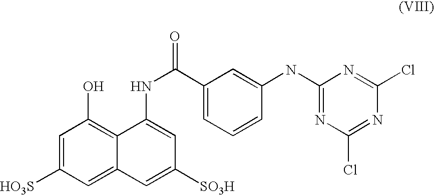
is obtained.
b) Condensation of Compound VIII with Anthranilic Acid

68,6 g of anthranilic acid is solved in water with sodiumhydroxide and added to the solution of compound (VIII). The temperature was increased to 45-47° C. and the pH keeped at 7.

After the termination of the reaction compound of formula (IX) is obtained. The compound was isolated by precipitation with sodiumchlorid. 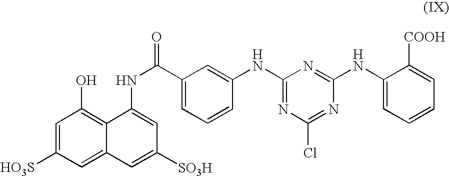
c) Condensation of IX with 3-Mercapto-1-Propanesulfonic acid

562.5 g of presscake of compound (IX) was solved in water and 86.5 g of 3-Mercapto-1-propanesulfonic acid were added. The temperature was increased to 60° C. and the ph keeped at 7.5-8.0

After 5 hours a compound of formula (X) 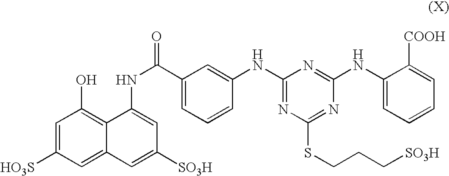
is salted out and filtered off.
d) Diazotization and Coupling with 2-Amino-1-Carboxybenzene-5-Sulfonic Acid

100 g of 2-amino-1-carboxybenzene-5-sulfonic acid in water are diazotizated at 0-5° C. and then added to a solution of 600 g presscake of compound (X) in water, keeping the pH at 7 by adding sodium hydroxide. After termination of this coupling the dyestuff of formula (XI) 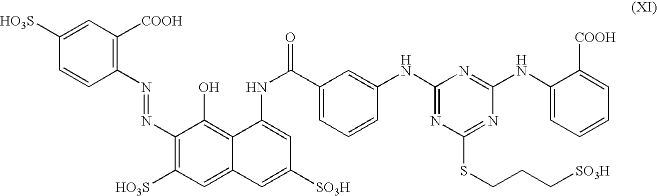
is salted out and filtred.

The presscake is washed with brine and then solved in water. The solution is desalted by membranfiltration at temperatures of 25-50° C. and pressures of 10-30 bars. After concentration and drying a dark red powder with a λₘₐₓ of 545.3 nm (measured in 2 wt-% sodium acetate in H₂O at room temperature) is obtained.

### Example 2

a) Condensation of Compound VIII with 2-Amino-1-Hydroxybenzene-4-Sulfonic Acid

95 g of 2-amino-1-hydroxybenzene-4-sulfonic acid is solved in water with sodiumhydroxide and added to a solution of compound (VIII), as produced in example 1. The temperature was increased to 30-35° C. and the pH keeped at 5. After the termination of this reaction the compound of formula (XII) 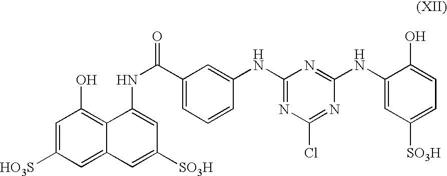
is obtained.
b) Condensation of Compound (XII) with N-Methyl-Taurine

600 g presscake of compound XII was solved in water and 70 g of N-methyl-taurine were added. The temperature was increased to 40-50° C. and the pH keeped at 8-8.5. A compound of formula (XIII) 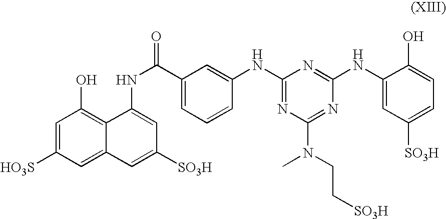
is obtained.
d) Diazotization and Coupling with 4-Amino-1-Methylbenzene-3-Sulfonic Acid

88 g of 4-amino-1-methylbenzene-3-sulfonic acid in water are diazotizated at 0-5° C. and then added to a solution of 580 g presscake of compound (XIII) in water, keeping the pH at 6-7 by adding sodium hydroxide. After termination of this coupling the dyestuff of formula (XIV) is salted out and filtred. The subsequent membrane filtration (at temperatures of 25-50° C. and pressures of 10-30 bars) and drying leads to a dark red powder. The dyestuff of formula (XIV) 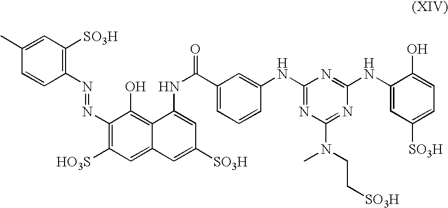
has a λₘₐₓ of 545.7 nm (measured in 2 wt-% sodium acetate in H₂O at room temperature), is obtained.

### Example 3

a) Condensation of N-Methyl-Taurine with Compound IX

138 g of the moist presscake consisting of the compound of formula (IX) is added to 500 ml of water. 17.1 g N-methyl-taurine is added and the temperature is increased to 60-65° C. and the pH-value is keeped at 8-8.5. After 2 hours a compound of formula (XV) 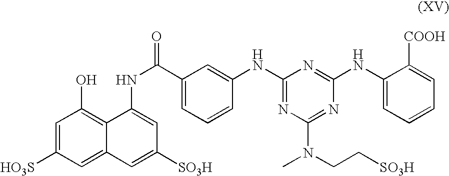
is obtained. This product is salted out and filtred off.
d) Diazotization and Coupling of Compound (XV) with 2-Naphthylamine1,5-Disulfonic Acid

37 g of 2-naphthylamine-1,5-disulfonic acid in water are diazotizated at 0-5° C. and then added to a solution of 250 g presscake of compound (XV) in water, keeping the pH at 7-8.5 by adding sodium hydroxide. After termination of this coupling the dyestuff of formula (XVI) is salted out and filtred. The subsequent membrane filtration (at temperatures of 25-50° C. and pressures of 10-30 bars) and drying leads to a dark red powder. The dyestuff of formula (XIV)

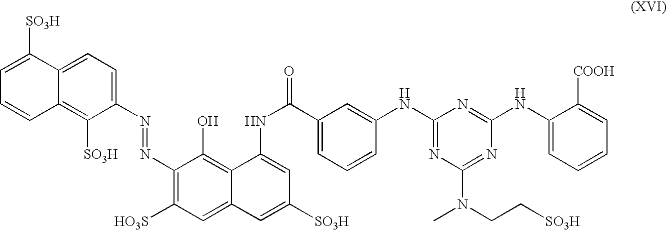

has a λₘₐₓ of 545.5 nm (measured in 2 wt-% sodium acetate in H₂O at room temperature),

TABLE 1
Examples 4-106
Analogously to Example 1 the following compounds according
to formula (XVII) were obtained.

| | |
|---|---|
| | (XVII) |
| 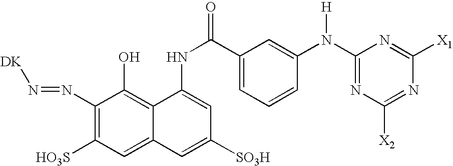 | |

| Exp. | DK | X₁ | X₂ | λₘₐₓ |
|---|---|---|---|---|
| 4 | 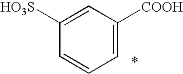 | 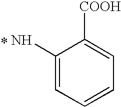 | 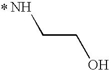 | 545.2 |
| 5 | 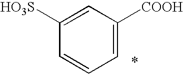 | 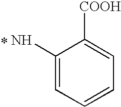 | 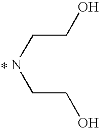 | 545.9 |
| 6 | 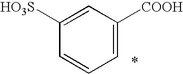 | 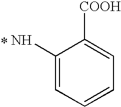 | 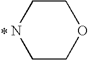 | 546.1 |
| 7 | 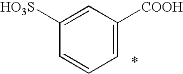 | 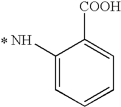 | 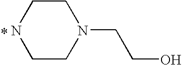 | 546.4 |
| 8 | 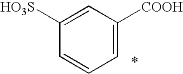 | 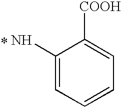 | 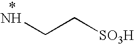 | 545.2 |
| 9 | 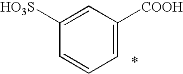 | 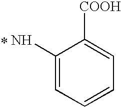 | 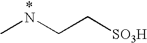 | 545.8 |
| 10 | 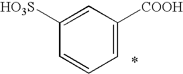 | 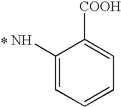 | 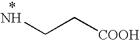 | 546.8 |
| 11 | 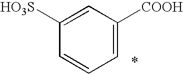 | 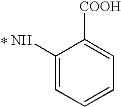 | 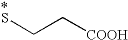 | 545.8 |
| 12 | 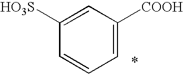 | 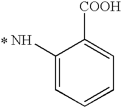 | 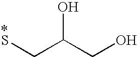 | 546.1 |
| 13 | 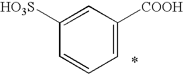 | 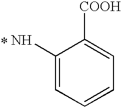 | 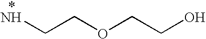 | 545.4 |
| 14 | 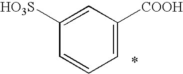 | 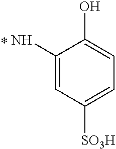 | 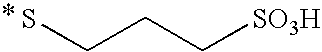 | 545.5 |
| 15 | 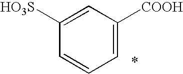 | 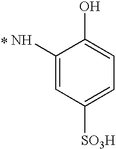 | 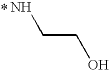 | 545.4 |
| 16 | 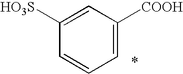 | 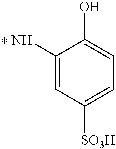 | 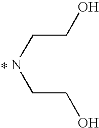 | 545.7 |
| 17 | 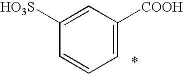 | 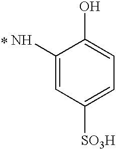 | 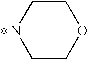 | 546.2 |
| 18 | 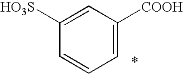 | 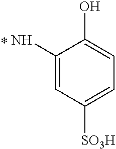 | 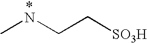 | 546.2 |
| 19 | 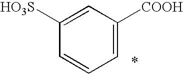 | 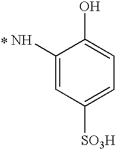 | 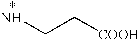 | 546.3 |
| 20 | 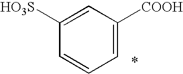 | 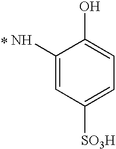 | 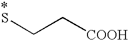 | 546.3 |
| 21 | 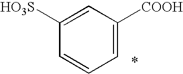 | 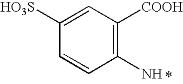 | 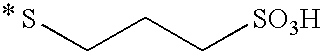 | 545.7 |
| 22 | 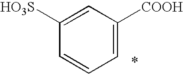 | 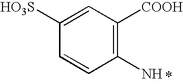 | 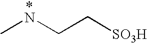 | 545.8 |
| 23 | 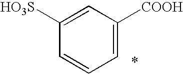 | 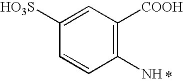 | 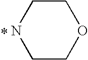 | 546.8 |
| 24 | 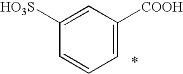 | 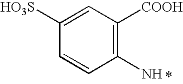 | 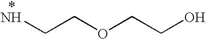 | 544.9 |
| 25 | 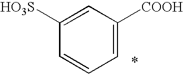 | 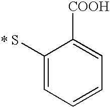 | 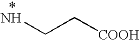 | 543.6 |
| 26 | 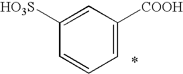 | 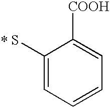 | 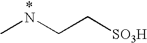 | 543.0 |
| 27 | 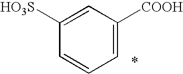 | 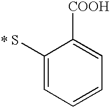 | 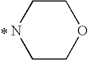 | 542.9 |
| 28 | 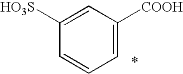 | 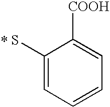 | 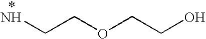 | 543.2 |
| 29 | 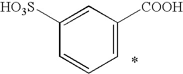 | 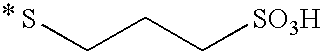 | 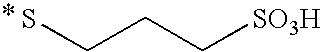 | 545.7 |
| 30 | 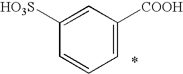 | 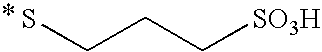 | 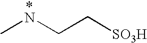 | 545.5 |
| 31 | 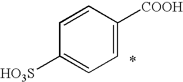 | 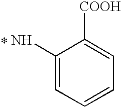 | 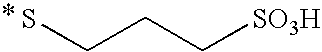 | 546.0 |
| 32 | 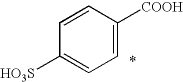 | 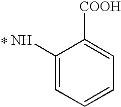 | 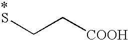 | 545.8 |
| 33 | 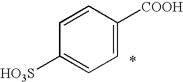 | 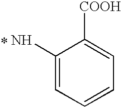 | 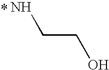 | 546.7 |
| 34 | 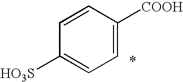 | 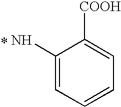 | 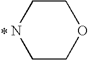 | 545.5 |
| 35 | 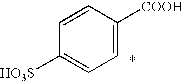 | 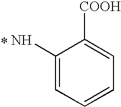 | 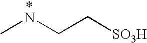 | 546.3 |
| 36 | 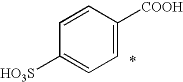 | 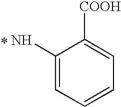 | 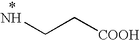 | 546.5 |
| 37 | 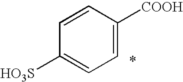 | 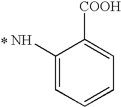 | 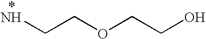 | 545.8 |
| 38 | 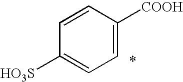 | 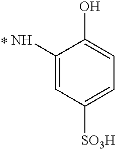 | 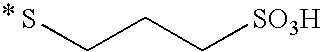 | 546.6 |
| 39 | 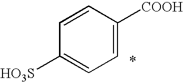 | 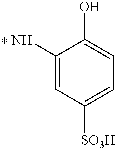 | 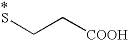 | 546.4 |
| 40 | 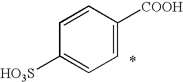 | 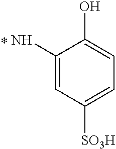 | 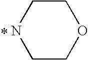 | 546.5 |
| 41 | 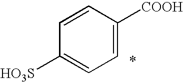 | 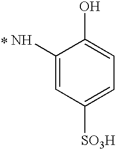 | 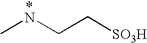 | 545.9 |
| 42 | 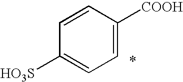 | 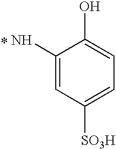 | 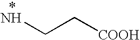 | 546.4 |
| 43 | 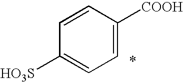 | 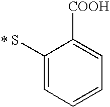 | 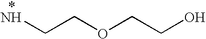 | 545.4 |
| 44 | 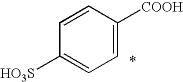 | 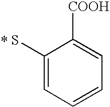 | 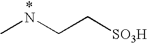 | 545.0 |
| 45 | 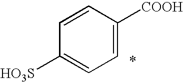 | 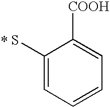 | 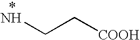 | 545.7 |
| 46 | 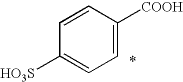 | 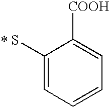 | 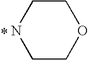 | 545.2 |
| 47 | 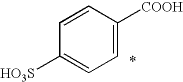 | 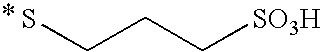 | 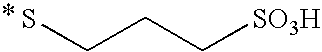 | 546.3 |
| 48 | 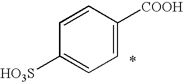 | 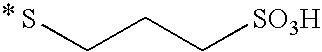 | 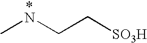 | 545.0 |
| 49 | 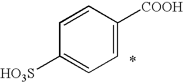 | 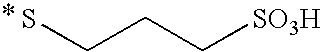 | 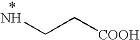 | 545.6 |
| 50 | 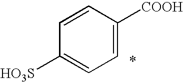 | 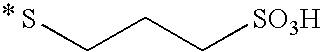 | 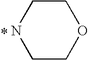 | 545.6 |
| 51 | 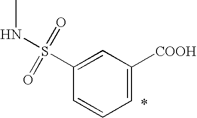 | 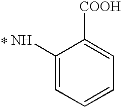 | 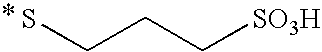 | 530.0 |
| 52 | 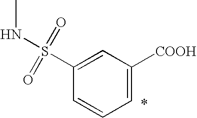 | 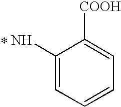 | 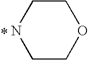 | 513.4 |
| 53 | 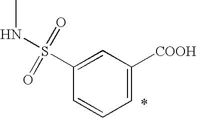 | 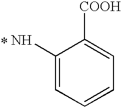 | 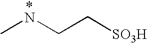 | 518.4 |
| 54 | 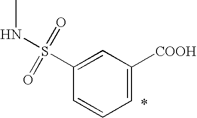 | 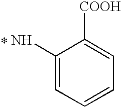 | 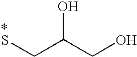 | 515.1 |
| 55 | 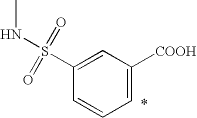 | 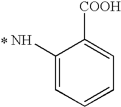 | 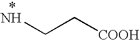 | 518.3 |
| 56 | 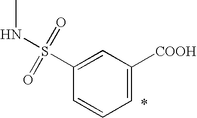 | 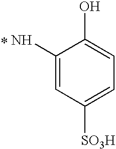 | 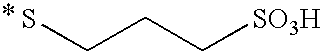 | 520.3 |
| 57 | 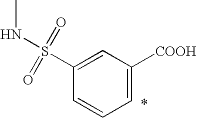 | 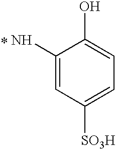 | 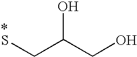 | 519.5 |
| 58 | 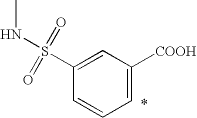 | 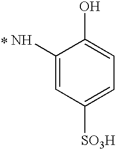 | 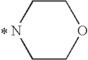 | 518.8 |
| 59 | 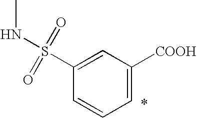 | 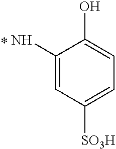 | 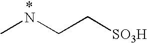 | 519.3 |
| 60 | 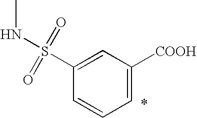 | 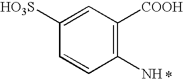 | 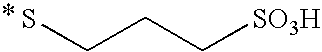 | 520.2 |
| 61 | 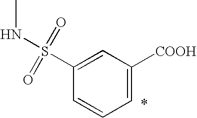 | 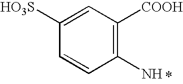 | 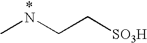 | 520.3 |
| 62 | 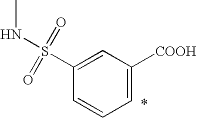 | 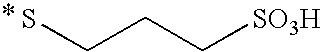 | 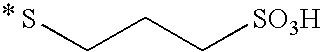 | 520.3 |
| 63 | 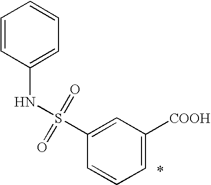 | 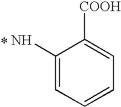 | 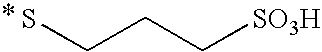 | 526.8 |
| 64 | 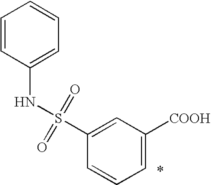 | 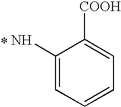 | 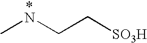 | 527.3 |
| 65 | 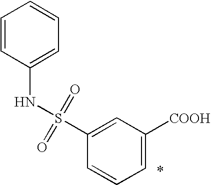 | 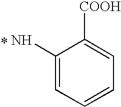 | 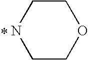 | 528.1 |
| 66 | 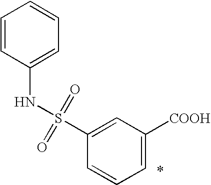 | 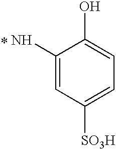 | 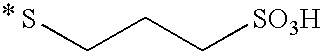 | 527.6 |
| 67 | 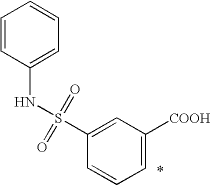 | 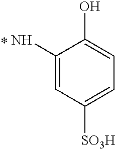 | 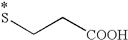 | 528.0 |
| 68 | 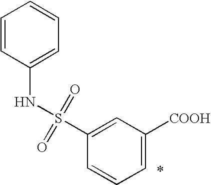 | 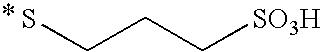 | 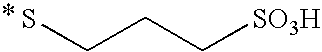 | 517.8 |
| 69 | 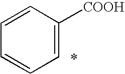 | 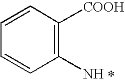 | 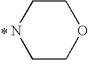 | 545.6 |
| 70 | 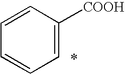 | 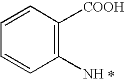 | 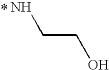 | 545.8 |
| 71 | 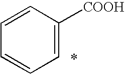 | 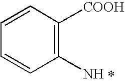 | 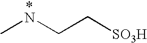 | 546.0 |
| 72 | 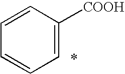 | 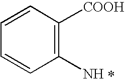 | 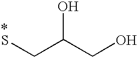 | 546.1 |
| 73 | 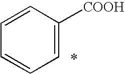 | 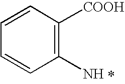 | 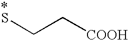 | 545.8 |
| 74 | 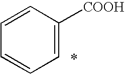 | 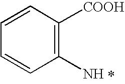 | 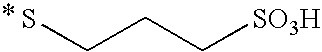 | 545.7 |
| 75 | 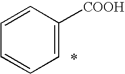 | 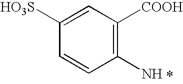 | 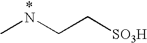 | 545.9 |
| 76 | 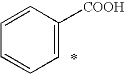 | 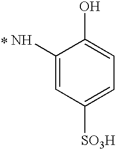 | 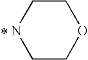 | 546.3 |
| 77 | 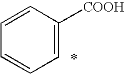 | 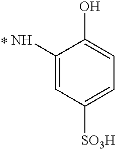 | 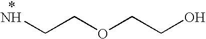 | 546.6 |
| 78 | 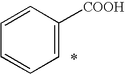 | 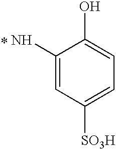 | 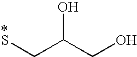 | 546.0 |
| 79 | 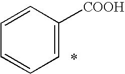 | 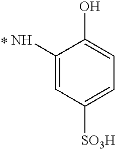 | 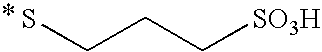 | 545.8 |
| 80 | 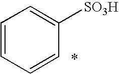 | 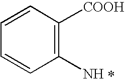 | 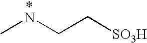 | 537.8 |
| 81 | 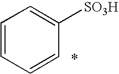 | 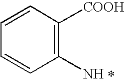 | 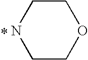 | 538.0 |
| 82 | 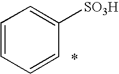 | 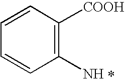 | 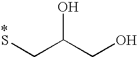 | 538.2 |
| 83 | 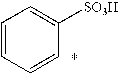 | 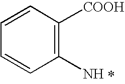 | 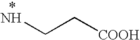 | 537.9 |
| 84 | 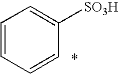 | 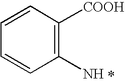 | 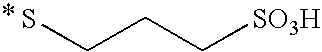 | 536.4 |
| 85 | 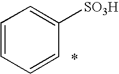 | 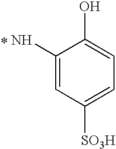 | 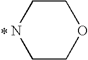 | 537.6 |
| 86 | 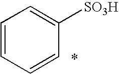 | 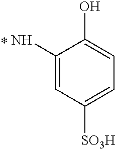 | 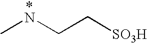 | 536.4 |
| 87 | 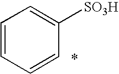 | 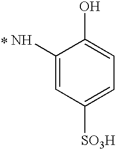 | 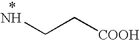 | 536.5 |
| 88 | 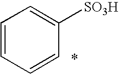 | 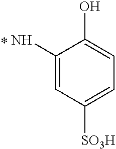 | 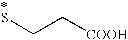 | 538.0 |
| 89 | 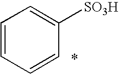 | 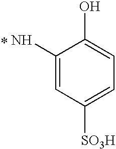 | 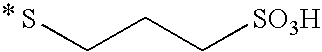 | 538.3 |
| 90 | 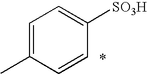 | 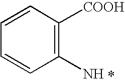 | 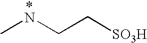 | 546.0 |
| 91 | 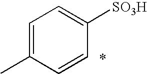 | 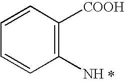 | 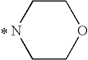 | 546.7 |
| 92 | 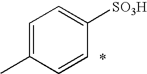 | 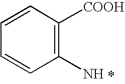 | 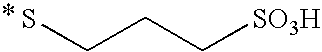 | 546.3 |
| 93 | 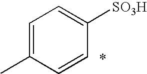 | 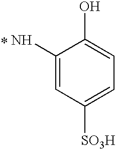 | 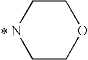 | 546.7 |
| 94 | 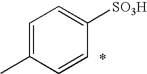 | 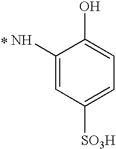 | 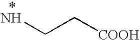 | 546.5 |
| 95 | 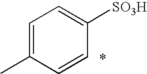 | 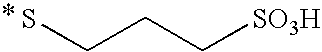 | 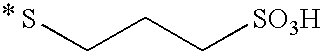 | 545.9 |
| 96 | 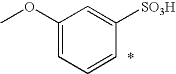 | 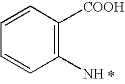 | 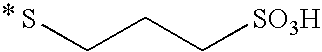 | 558.9 |
| 97 | 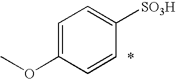 | 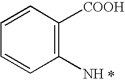 | 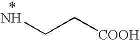 | 559.0 |
| 98 | 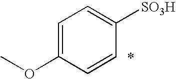 | 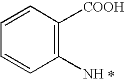 | 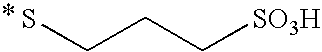 | 556.2 |
| 99 | 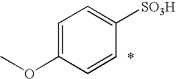 | 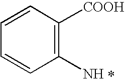 | 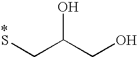 | 562.1 |
| 100 | 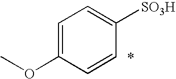 | 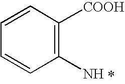 | 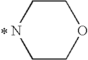 | 560.1 |
| 101 | 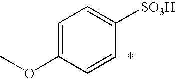 | 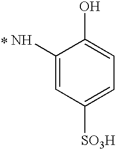 | 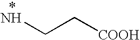 | 559.0 |
| 102 | 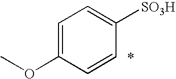 | 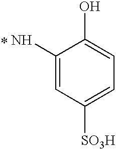 | 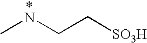 | 559.4 |
| 103 | 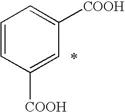 | 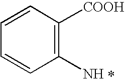 | 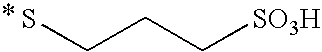 | 547.3 |
| 104 | 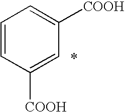 | 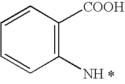 | 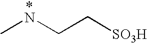 | 547.4 |
| 105 | 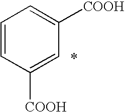 | 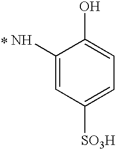 | 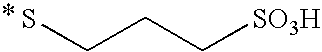 | 547.7 |
| 106 | 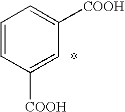 | 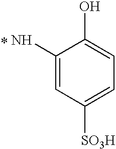 | 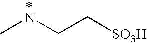 | 547.0 |

*λₘₐₓ-values are measured in 2 wt-‰ sodium acetate in H₂O at room temperature)

TABLE 2
Examples 107-118
Analogously to Example 3 the following compounds according
to formula (XVIII) were obtained.

| | |
|---|---|
| | (XVIII) |
| 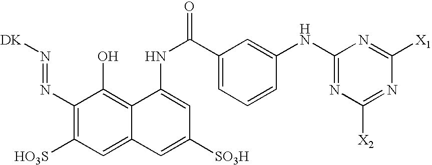 | |

| Exp. | DK | X₁ | X₂ | λₘₐₓ* [nm] |
|---|---|---|---|---|
| 107 | 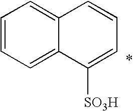 | 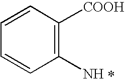 | 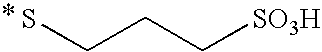 | 550.8 |
| 108 | 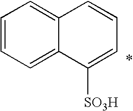 | 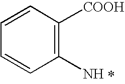 | 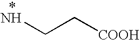 | 549.8 |
| 109 | 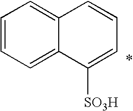 | 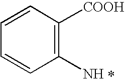 | 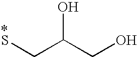 | 551.1 |
| 110 | 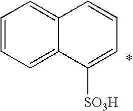 | 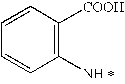 | 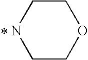 | 550.5 |
| 111 | 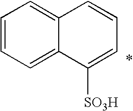 | 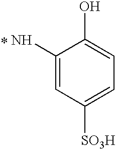 | 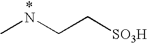 | 553.2 |
| 112 | 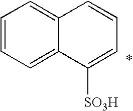 | 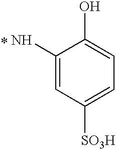 | 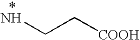 | 550.8 |
| 113 | 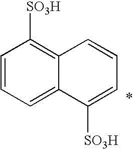 | 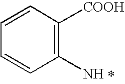 | 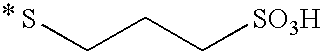 | 546.6 |
| 114 | 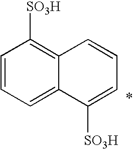 | 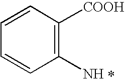 | 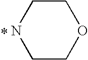 | 545.8 |
| 115 | 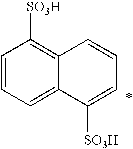 | 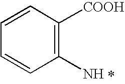 | 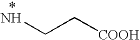 | 545.1 |
| 116 | 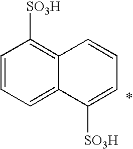 | 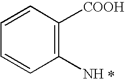 | 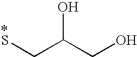 | 546.8 |
| 117 | 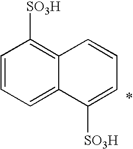 | 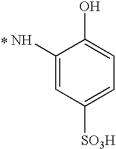 | 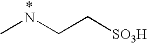 | 545.5 |
| 118 | 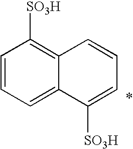 | 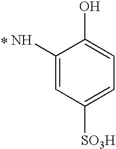 | 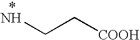 | 544.3 |

*λₘₐₓ-values are measured in 2 wt-‰ sodium acetate in H₂O at room temperature)

Dyestuff Mixtures

The following dyestuff mixtures are used to formulate a composition for printing recording materials as described above. Such a composition comprises
1) a dyestuff or a mixture of dyestuffs as defined above and
2) water or a medium including a mixture of water and an organic solvent, an anhydrous organic solvent or a solid having a low melting point, and
3) optionally further additives.

### Example 119 (Dyestuff Mixture 1)

| | |
|---|---|
| 90 wt-% | of the Example 1 and |
| 10 wt-% | of C.I. Acid Red 52. |

This mixture is used to formulate a composition comprising

| | |
|---|---|
| 2.5 wt-% | of Example 119 (dyestuff Mixture 1) |
| 15 wt-% | of N-methyl-pyrrolidone |
| 82.5 wt-% | of water. |

This mixture is further used to formulate a composition comprising

| | |
|---|---|
| 2.5 wt-% | of Example 119 (dyestuff Mixture 1) |
| 20 wt-% | of 1,2 propyleneglycol |
| 77.5 wt-% | of water. |

TABLE 3
Examples 120-123

| | | wt-% of | | wt-% of |
|---|---|---|---|---|
| Exp. | Compound 1 | Compound 1 | Compound 2 | Compound 2 |
| 120 | Example 1 | 80 | C.I. Acid Red 52 | 20 |
| 121 | Example 1 | 70 | C.I. Acid Red 289 | 30 |
| 122 | Example 2 | 80 | C.I. Acid Red 52 | 20 |
| 123 | Example 2 | 70 | C.I. Acid Red 289 | 30 |

Compound 1 is a compound according to formula (I) or a mixture thereof.
Compound 2 is a dyestuff named by its Colour Index name.

All Examples (1-118) as well as the compounds according to formula (I) and mixtures thereof are suitable as Compound 1.

All compounds C.I. Acid Red and C.I. Direct Red compounds as well as mixtures thereof, which are listed in the description are suitable as Compound 2

### APPLICATION EXAMPLES

### Application Example A

An ink consisting of 2.5 parts of the dyestuff of Example 1 in 97.5 parts of a mixture of water and N-methylpyrrolidone where the ratio of water to N-methylpyrrolidone is 85:15 is introduced into an HP 880C DeskJet Printer and printed onto an A4 Epson Premium Glossy Photo Paper; S041287 (HP and DeskJet are registered trademarks of Hewlett-Packard, Palo Alto, Calif., USA; Epson is a registered trademark of Seiko Epson Kabushiki Kaisha.). The magenta prints thus obtained have very good ozon- and lightfastnesses.

This application example can be used in a similar manner for all the examples of the present application. Similarly, mixtures of individual dyes can be used. The prints thus obtained have very good ozon- and lightfastnesses.

### Application Example B

An ink consisting of 2.5 parts of the dyestuff of Example 2 in 97.5 parts of a mixture of water, propylene glycol and isopropanole where the ratio of water: propylene glycol and isopropanole is 90:5:5 is introduced into an HP 880C DeskJet Printer and printed onto A4 Epson Premium Glossy Photo Paper; S041287 (HP and DeskJet are registered trademarks of Hewlett-Packard, Palo Alto, Calif., USA; Epson is a registered trademark of Seiko Epson Kabushiki Kaisha.). The magenta prints thus obtained have very good ozon- and lightfastnesses.

This application example can be used in a similar manner for all the examples of the present application. Similarly, mixtures of individual dyes can be used. The prints thus obtained have good fastnesses.

### Application Example C

An ink consisting of 2.5 parts of the dyestuff of Example 3 in 97.5 parts of a mixture of water and N-methylpyrrolidone where the ratio of water to N-methylpyrrolidone is 85:15 is introduced into an HP 880C DeskJet Printer and printed onto an A4 Epson Premium Glossy Photo Paper; S041287 (HP and DeskJet are registered trademarks of Hewlett-Packard, Palo Alto, Calif., USA; Epson is a registered trademark of Seiko Epson Kabushiki Kaisha.). The magenta prints thus obtained have good fastnesses especially ozon- and lightfastness.

This application example can be used in a similar manner for all the examples of the present application. Similarly, mixtures of individual dyes can be used. The prints thus obtained have good fastnesses and the color is brilliant.

### Application Example D

An ink consisting of 2.5 parts of dyestuff of Example 14 in 97.5 parts of a mixture of water, propylene glycol and isopropanole where the ratio of water:propylene glycol and isopropanole is 90:5:5 is introduced into an HP 880C DeskJet Printer and printed onto an A4 Epson Premium Glossy Photo Paper; S041287 (HP and DeskJet are registered trademarks of Hewlett-Packard, Palo Alto, Calif., USA; Epson is a registered trademark of Seiko Epson Kabushiki Kaisha.). The magenta prints thus obtained have good fastnesses especially ozon- and lightfastness and the color is brilliant.

This application example can be used in a similar manner for all the examples of the present application. Similarly, mixtures of individual dyes can be used. The prints thus obtained have good fastnesses and the colors is brilliant.

### Application Example E

An ink consisting of 2.5 parts of the dyestuff of Example 42 in 97.5 parts of a mixture of water and N-methylpyrrolidone where the ratio of water to N-methylpyrrolidone is 85:15 is introduced into an HP 880C DeskJet Printer and printed onto an A4 Epson Premium Glossy Photo Paper; S041287 (HP and DeskJet are registered trademarks of Hewlett-Packard, Palo Alto, Calif., USA; Epson is a registered trademark of Seiko Epson Kabushiki Kaisha.). The magenta prints thus obtained have very good ozon- and lightfastnesses.

This application example can be used in a similar manner for all the examples of the present application. Similarly, mixtures of individual dyes can be used. The prints thus obtained have good fastnesses.

### Application Example F

An ink consisting of 2.5 parts of the dyestuff of Example 94 in 97.5 parts of a mixture of water and N-methylpyrrolidone where the ratio of water to N-methylpyrrolidone is 85:15 is introduced into an HP 880C DeskJet Printer and printed onto an A4 Epson Premium Glossy Photo Paper; S041287 (HP and DeskJet are registered trademarks of Hewlett-Packard, Palo Alto, Calif., USA; Epson is a registered trademark of Seiko Epson Kabushiki Kaisha.). The magenta prints thus obtained have very good ozon- and lightfastnesses.

This application example can be used in a similar manner for all the examples of the present application. Similarly, mixtures of individual dyes can be used. The prints thus obtained have good fastnesses

### Application Example G

An ink consisting of 2.5 parts of the mixture of Example 119 (dye mixture 1) in 97.5 parts of a mixture of water and N-methylpyrrolidone where the ratio of water to N-methylpyrrolidone is 85:15 is introduced into an HP 880C DeskJet Printer and printed onto an A4 Epson Premium Glossy Photo Paper; S041287 (HP and DeskJet are registered trademarks of Hewlett-Packard, Palo Alto, Calif., USA; Epson is a registered trademark of Seiko Epson Kabushiki Kaisha.). The magenta prints thus obtained have very good ozon- and lightfastnesses.

This application example can be used in a similar manner for all the examples of the present application. Similarly, mixtures of individual dyes can be used. The prints thus obtained have good fastnesses

### Application Example H

An ink consisting of 2.5 parts of the mixture of Example 120 (dye mixture 2) in 97.5 parts of a mixture of water and 2-pyrrolidone where the ratio of water to N-methylpyrrolidone is 85:15 is introduced into an HP 880C DeskJet Printer and printed onto an A4 Epson Premium Glossy Photo Paper; S041287 (HP and DeskJet are registered trademarks of Hewlett-Packard, Palo Alto, Calif., USA; Epson is a registered trademark of Seiko Epson Kabushiki Kaisha.). The magenta prints thus obtained have very good ozon- and lightfastnesses.

This application example can be used in a similar manner for all the examples of the present application. Similarly, mixtures of individual dyes can be used. The prints thus obtained have good fastnesses

1. A dyestuff of formula (I) 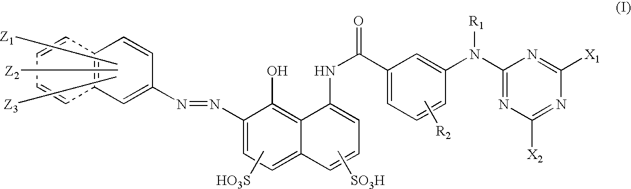
wherein
R₁ is H; C₁₋₄alkyl; substituted C₁₋₄alkyl; phenyl or substituted phenyl,
R₂ is H; C₁₋₄alkyl; substituted C₁₋₄alkyl; C₁₋₄alkoxy; —COOH; —COOCH₃; —CF₃; —SO₃H; —CN or SO₂NHR6,
wherein R₆ is H, C₁₋₄Alkyl, phenyl or substituted phenyl
X₁ is NR₃R₄; SR₅; OH;
X₂ is NR₃R₄; SR₅; OH;
wherein
R₃ is H, C₁₋₄alkyl; substituted C₁₋₄alkyl; substituted phenyl, naphthyl or substituted naphthyl
R₄ is H; C₁₋₄alkyl; substituted C₁₋₄alkyl; substituted phenyl, naphthyl or substituted naphthyl
or R₃ and R₄ form 5- or 6-membered ring containing one or two hetero atoms, in addition to N, O or S,which heterocyclic ring is unsubstituted or substituted by one or two C₁₋₄alkyl groups
R₅ is C₁₋₄alkyl; substituted C₁₋₄alkyl; phenyl or substituted phenyl and X₁ is not the same as X₂ unless X₁ or X₂ is SR₅ or OH;
and
Z₁ is H; C₁₋₄alkyl; substituted C₁₋₄alkyl; C₁₋₄alkoxy; —OH; —COOH; —COOCH₃; —
CF₃; —SO₃H ; amino; alkylamino, —CN or SO₂NHR′₆, wherein R′₆ is H, C₁₋₄ alkyl, phenyl or substituted phenyl
Z₂ is H; C₁₋₄alkyl; substituted C₁₋₄alkyl; C₁₋₄alkoxy; OH; COOH; —SO₃H
Z₃ is H, C₁₋₄alkyl; substituted C₁₋₄alkyl; C₁₋₄alkoxy; OH; COOH; —SO₃H
wherein the dyestuff of Formula I is in the free acid or in salt form, as well as mixtures thereof.

2. A dyestuff according to claim 1 wherein
R₁ is H; C₁₋₄alkyl; substituted C₁₋₄alkyl,
R₂ is H; C₁₋₄alkyl; substituted C₁₋₄alkyl; C₁₋₄alkoxy; —COOH or —SO₃H
X₁ is NR₃R₄; SR₅; OH;
X₂ is NR₃R₄; SR5; OH;
wherein
R₃ is H, C₁₋₄alkyl; substituted C₁₋₄alkyl; phenyl or substituted phenyl, naphthyl or substituted naphthyl
R₄ is H; C₁₋₄alkyl; substituted C₁₋₄alkyl; phenyl or substituted phenyl, naphthyl or substituted naphthyl or
R₃ and R₄ form a 5- or 6-membered ring containing one or two hetero atoms, in addition to N, O or S, which heterocyclic ring is unsubstituted or substituted by one or two C₁₋₄alkyl groups
R₅ is C₁₋₄alkyl; substituted C₁₋₄alkyl; phenyl or substituted phenyl and X₁ is not the same as X₂ unless X₁ or X₂ signifies SR₅ or OH;
and
Z₁ is H; C₁₋₄alkyl; substituted C₁₋₄alkyl; C₁₋₄alkoxy; —OH; —COOH; —COOCH₃; —CF₃; —SO₃H ; amino; alkylamino, —CN or SO₂NHR′₆,
wherein R′₆ is H, C₁₋₄ alkyl, phenyl or substituted phenyl
Z₂ is H; C₁₋₄alkyl; substituted C₁₋₄alkyl; C₁₋₄alkoxy; OH; COOH; —SO₃H
Z₃ is H, C₁₋₄alkyl; substituted C₁₋₄alkyl; C₁₋₄alkoxy; OH; COOH; —SO₃H
wherein the dyestuff of Formula I is in the free acid or in salt form, as well as mixtures thereof.

3. A dyestuff according to claim 2 of formula (Ia) 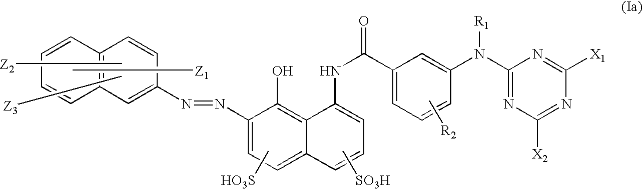
wherein
R₁ is H; C₁₋₄alkyl; substituted C₁₋₄alkyl,
R₂ is H; C₁₋₄alkyl; substituted C₁₋₄alkyl; C₁₋₄alkoxy; —COOH or —SO₃H
X₁ is NR₃R₄; SR₅; OH;
X₂ is NR₃R₄; SR; OH;
wherein
R₃ is H, C₁₋₄alkyl; substituted C₁₋₄alkyl; phenyl or substituted phenyl, naphthyl or substituted naphthyl
R₄ is H; C₁₋₄alkyl; substituted C₁₋₄alkyl; phenyl or substituted phenyl, naphthyl or substituted naphthyl or
R₃ and R₄ form a 5- or 6-membered ring containing one or two hetero atoms, in addition to N, O or S, which heterocyclic ring is unsubstituted or substituted by one or two C₁₋₄alkyl groups
R₅ is C₁₋₄alkyl; substituted C₁₋₄alkyl; phenyl or substituted phenyl and X₁ is not the same as X₂ unless X₁ or X₂ is SR₅ or OH;
and
Z₁ is H; C₁₋₄alkyl; substituted C₁₋₄alkyl; C₁₋₄alkoxy; —OH; —COOH; —COOCH₃; —CF₃; —SO₃H ; amino; alkylamino, —CN or SO₂NHR′₆, wherein R′₆ is H, C₁₋₄ alkyl, phenyl or substituted phenyl
Z₂ is H; C₁₋₄alkyl; substituted C₁₋₄alkyl; C₁₋₄alkoxy; OH; COOH; —SO₃H
Z₃ is H, C₁₋₄alkyl; substituted C₁₋₄alkyl; C₁₋₄alkoxy; OH; COOH; —SO₃H wherein the dyestuff of Formula Ia is in the free acid or in salt form, as well as mixtures thereof.

4. A dyestuff according to claim 2 of formula (Ib) 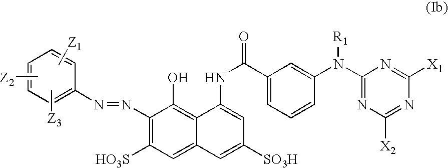
wherein
R₁ is H; C₁₋₄alkyl; substituted C₁₋₄alkyl,
R₂ is H; C₁₋₄alkyl; substituted C₁₋₄alkyl; C₁₋₄alkoxy; —COOH or —SO₃H
X₁ is NR₃R₄; S₅; OH;
X₂ is NR₃R₄; SR₅; OH;
wherein
R₃ is H, C₁₋₄alkyl; substituted C₁₋₄alkyl; phenyl or substituted phenyl, naphthyl or substituted naphthyl
R₄ is H; C₁₋₄alkyl; substituted C₁₋₄alkyl; phenyl or substituted phenyl, naphthyl or substituted naphthyl or
R₃ and R₄ form a 5- or 6-membered ring containing one or two hetero atoms, in addition to N, O or S, which heterocyclic ring is unsubstituted or substituted by one or two C₁₋₄alkyl groups
R₅ is C₁₋₄alkyl; substituted C₁₋₄alkyl; phenyl or substituted phenyl and X₁ is not the same as X₂ unless X₁ or X₂ is SR₅or OH; and
Z₁ is H; C₁₋₄alkyl; substituted C₁₋₄alkyl; C₁₋₄alkoxy; —OH; —COOH; —COOCH₃; —CF₃; —SO₃H ; amino; alkylamino, —CN or SO₂NHR′₆, wherein R′₆ is H, C₁₋₄ alkyl, phenyl or substituted phenyl
Z₂ is H; C₁₋₄alkyl; substituted C₁₋₄alkyl; C₁₋₄alkoxy; OH; COOH; —SO₃H
Z₃ is H, C₁₋₄alkyl; substituted C₁₋₄alkyl; C₁₋₄alkoxy; OH; COOH; —SO₃H wherein the dyestuff of Formula Ib is in the free acid or in salt form, as well as mixtures thereof

5. A process for the preparation of a compound according to the formula (I) comprising the steps of i) reacting a compound of formula (II) 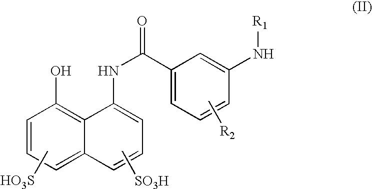
wherein
R₁ is H; C₁₋₄alkyl: substituted C₁₋₄alkyl,
R₂ is H; C₁₋₄alkyl; substituted C₁₋₄alkyl; C₁₋₄alkoxy; —COOH or —SO₃H with a compound of formula (Ill) 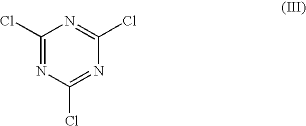
resulting in compound according to formula (IV) 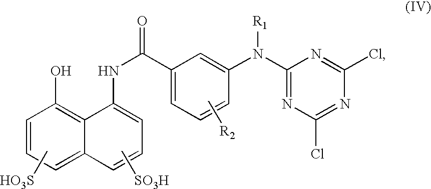
ii) reacting the compound of formula (IV) with one part of a compound of formula HX₁ wherein
X₁ is NR₃R₄; SR₅; OH;
resulting in compound of formula (V) 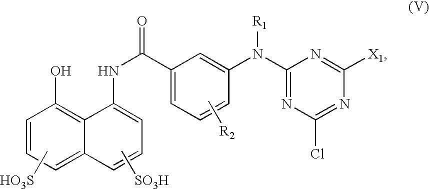
iii)reacting the compound of formula (V) with a compound of formula HX₂ wherein
X₂ is NR₃R_{b 4;} SR₅; OH:
and X₁ is not the same as X₂ unless X₁ or X₂ is Sr₅ or OH: resulting in compound of formula (VI) 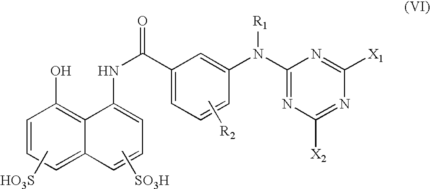
iv) coupling the compound of formula (VI) with the diazonium salt of a compound of formula (VII) 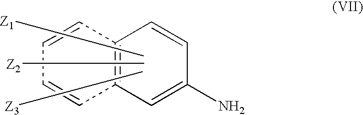
wherein
Z₁ is H; C₁₋₄alklyl substituted C₁₋₄alkyl; C₁₋₄alkoxy: —OH; —COOH; —COOCH₃: —CF₃: —SO₃H: amino, alkylamino, —CN or SO₂NHR′₆, wherein R′₆ is H, C₁₋₄alkyl, phenyl or substituted phenyl
Z₂ is H; C₁₋₄alkyl; substituted C₁₋₄alkyl: C₁₋₄alkoxy; OH; COOH; —SO₃H
Z₃ is H, C₁₋₄alkyl; substituted C₁₋₄alkyl; C₁₋₄alkoxy; OH; COOH; —SO₃H
resulting in the dyestuff of formula (I)

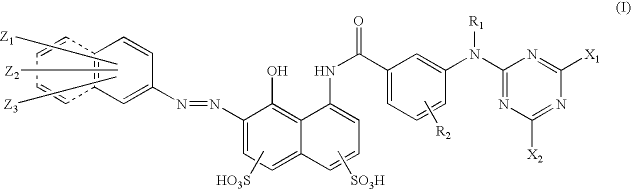

6. An Ink Jet Ink comprising at least one compound according to claim 1.

7. An Ink Jet Ink according to claim 6 wherein the total content of salts is less than 0.5% by weight, based on the total weight of the dyes.

8. An inkjet printing process for printing a recording material or dyeing a substrate comprising cellulose or both, comprising the step of contacting at least one dyestuff according to claim 1 with the substrate comprising cellulose.

9. A process according to claim 8 wherein the recording material is paper or a papery substrate.

10. A recording material or a papery substrate or a substrate comprising cellulose printed or dyed with a compound according to claim 1.
